(19) United States
(12) Patent Application Publication
(10) Pub. No.: US 2007/0161657 A1
(43) Pub. Date: Jul. 12, 2007

(54) Crystalline N-(4-(4-aminothieno[2,3-d]pyrimidin-5-yl)phenyl)-N'-(2-fluoro-5-(trifluoromethyl)phenyl)urea hydrobromide

(76) Inventors: Sean M. Mellican, Gurnee, IL (US); Cathie L. Linton, Waukegan, IL (US); Jianzhang Mei, Lake Forest, IL (US); Jason S. Tedrow, Santa Monica, CA (US); Nahathai Charukamnoetkanok, Pittsburgh, PA (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/642,396

(22) Filed: Dec. 20, 2006

Related U.S. Application Data
(60) Provisional application No. 60/754,343, filed on Dec. 28, 2005.

(51) Int. Cl. A61K 31/519; C07D 498/02

(52) U.S. Cl. 514/260.1; 544/278

(57) ABSTRACT

A crystalline N-(4-(4-aminothieno[2,3-d]pyrimidin-5-yl)phenyl)-N′-[2-fluoro-5-(trifluoromethyl)phenyl)urea hydrobromide, ways to make it, compositions comprising it, and methods of treatment using it are disclosed.

This application claims priority to U.S. Provisional Application Ser. No. 60/754,343, Dec. 28, 2005.

### FIELD OF THE INVENTION

This invention pertains to a crystalline N-(4-(4-aminothieno[2,3-d]pyrimidin-5-yl)phenyl)-N′-(2-fluoro-5-(trifluoromethyl)phenyl)urea hydrobromide, ways to make it, compositions comprising it and methods of treatment using it.

### BACKGROUND OF THE INVENTION

The compound N-(4-(4-aminothieno[2,3-d]pyrimidin-5-yl)phenyl)-N′-(2-fluoro-5-(trifluoromethyl)phenyl)urea is useful for treating diseases caused or exascerbated by upregulation or overexpression of protein tyrosine kinases.

Because the crystallinity of salts of compounds may effect, among other physical and mechanical properties, their solubility, dissolution rate, hardness, compressability and melting point, there is an existing need in the process and therapeutic arts for identification of crystalline salts of N-(4-(4-aminothieno[2,3-d]pyrimidin-5-yl)phenyl)-N′-(2-fluoro-5-(trifluoromethyl)phenyl)urea and ways to reproducibly make them.

### SUMMARY OF THE INVENTION

One embodiment of this invention, therefore, pertains to crystalline N-(4-(4-aminothieno[2,3-d]pyrimidin-5-yl)phenyl)-N′-(2-fluoro-5-(trifluoromethyl)phenyl)urea hydrobromide characterized, when measured at about 25° C. with radiation at 1.54178 Å, by a powder diffraction pattern with at least three peaks having respective 2θ values of about 6.1°, 8.4°, 10.4°, 12.5°, 13.3°, 14.3°, 15.9°, 17.5°, 19.2° or 21.5°.

Another embodiment pertains to crystalline N-(4-(4-aminothieno[2,3-d]pyrimidin-5-yl)phenyl)-N′-(2-fluoro-5-(trifluoromethyl)phenyl)urea hydrobromide having substantial crystalline purity and characterized, when measured at about 25° C. with radiation at 1.54178 Å, by a powder diffraction pattern with at least three peaks having respective 2θ values of about 6.1°, 8.4°, 10.4°, 12.5°, 13.3°, 14.3°, 15.9°, 17.5°, 19.2° or 21.5°.

Still another embodiment pertains to a composition comprising an excipient and crystalline N-(4-(4-aminothieno[2,3-d]pyrimidin-5-yl)phenyl)-N′-(2-fluoro-5-(trifluoromethyl)phenyl)urea hydrobromide characterized, when measured at about 25° C. with radiation at 1.54178 Å, by a powder diffraction pattern with at least three peaks having respective 2θ values of about 6.1°, 8.4°, 10.4°, 12.5°, 13.3°, 14.3°, 15.9°, 17.5°, 19.2° or 21.5°.

Still another embodiment pertains to a method or treating a patient having a disease caused or exascerbated by upregulation or overexpression of protein tyrosine kinases comprising administering thereto a therapeutically effective amount of crystalline N-(4-(4-aminothieno[2,3-d]pyrimidin-5-yl)phenyl)-N′-(2-fluoro-5-(trifluoromethyl)phenyl)urea hydrobromide characterized, when measured at about 25° C. with radiation at 1.54178 Å, by a powder diffraction pattern with at least three peaks having respective 2θ values of about 6.1°, 8.4°, 10.4°, 12.5°, 13.3°, 14.3°, 15.9°, 17.5°, 19.2° or 21.5°.

Still another embodiment pertains to a process for making a crystalline N-(4-(4-aminothieno[2,3-d]pyrimidin-5-yl)phenyl)-N′-(2-fluoro-5-(trifluoromethyl)phenyl)urea hydrobromide, said process comprising:

providing a mixture comprising N-(4-(4-aminothieno[2,3-d]pyrimidin-5-yl)phenyl)-N′-(2-fluoro-5-(trifluoromethyl)phenyl)urea and solvent, wherein said N-(4-(4-aminothieno[2,3-d]pyrimidin-5-yl)phenyl)-N′-(2-fluoro-5-(trifluoromethyl)phenyl)urea is completely dissolved in said solvent;

causing crystalline N-(4-(4-aminothieno[2,3-d]pyrimidin-5-yl)phenyl)-N′-(2-fluoro-5-(trifluoromethyl)phenyl)urea hydrobromide to exist in said mixture, said N-(4-(4-aminothieno[2,3-d]pyrimidin-5-yl)phenyl)-N′-(2-fluoro-5-(trifluoromethyl)phenyl)urea hydrobromide, when isolated, characterized, when measured at about 25° C. with radiation at 1.54178 Å, by a powder diffraction pattern with at least three peaks having respective 2θ values of about 6.1°, 8.4°, 10.4°, 12.5°, 13.3°, 14.3°, 15.9°, 17.5°, 19.2° or 21.5°; and

isolating said crystalline N-(4-(4-aminothieno[2,3-d]pyrimidin-5-yl)phenyl)-N′-(2-fluoro-5-(trifluoromethyl)phenyl)urea hydrobromide.

Still another embodiment pertains to N-(4-(4-aminothieno[2,3-d]pyrimidin-5-yl)phenyl)-N′-(2-fluoro-5-(trifluoromethyl)phenyl)urea hydrobromide prepared by the foregoing process.

### DETAILED DESCRIPTION OF THE INVENTION

This invention pertains to discovery of a new crystalline form of N-(4-(4-aminothieno[2,3-d]pyrimidin-5-yl)phenyl)-N′-(2-fluoro-5-(trifluoromethyl)phenyl)urea hydrobromide, ways to characterize it, compositions containing it and methods of treating diseases caused or exascerbated by upregulation or overexpression of protein tyrosine kinases using it.

The term “diseases caused or exascerbated by upregulation or overexpression of protein tyrosine kinases,” as used herein, means angiogenic diseases (e.g. diabetic retinopathy, retinopathy of prematurity, choroidal neovascularization due to age-related macular degeneration, infantile hemangiomas, cancer (lung, breast, stomach, bladder, colon, pancreatic, ovarian, prostate and rectal cancer and hematopoietic malignancies (leukemia and lymphoma), glioblastoma, infantile hemangioma)) (Lab. Investig. (1992), 67(4), 519-528; Anat. Rec. (1997), 249(1), 63-73; Int. J. Cancer (1995), 63(5), 694-701; Vasc. Biol. (1995), 15(11), 1857-6)), pulmonary hypertension in patients with thromboembolic disease (J. Thorac. Cardiovasc. Surg. 2001, 122 (1), 65-73) and autoimmune diseases (psoriasis, kidney rejection, graft versus host disease).

The term “amorphous,” as used herein, means a supercooled liquid substance or a viscous liquid which may appear solid but is neither crystalline nor microcrystalline. Amorphous substances do not have a melting point but soften or flow above a certain temperature known as the glass transition temperature.

The term “crystalline,” as used herein, means having a regularly repeating arrangement of molecules which is maintained over a long range or external face planes.

The term “N-(4-(4-aminothieno[2,3-d]pyrimidin-5-yl)phenyl)-N′-(2-fluoro-5-(trifluoromethyl)phenyl)urea hydrobromide,” as used herein, means an amorphous form of N-(4-(4-aminothieno[2,3-d]pyrimidin-5-yl)phenyl)-N′-(2-fluoro-5-(trifluoromethyl)phenyl)urea hydrobromide, microcrystalline N-(4-(4-aminothieno[2,3-d]pyrimidin-5-yl)phenyl)-N′-(2-fluoro-5-(trifluoromethyl)phenyl)urea hydrochloride, N-(4-(4-aminothieno[2,3-d]pyrimidin-5-yl)phenyl)-N′-(2-fluoro-5-(trifluoromethyl)phenyl)urea hydrobromide in solution, a particular crystalline form of N-(4-(4-aminothieno[2,3-d]pyrimidin-5-yl)phenyl)-N′-(2-fluoro-5-(trifluoromethyl)phenyl)urea hydrobromide or a mixture thereof.

The term “crystalline N-(4-(4-aminothieno[2,3-d]pyrimidin-5-yl)phenyl)-N′-(2-fluoro-5-(trifluoromethyl)phenyl)urea hydrobromide,” as used herein, means a particular crystalline form of N-(4-(4-aminothieno[2,3-d]pyrimidin-5-yl)phenyl)-N′-(2-fluoro-5-(trifluoromethyl)phenyl)urea hydrobromide such as the crystalline N-(4-(4-aminothieno[2,3-d]pyrimidin-5-yl)phenyl)-N′-(2-fluoro-5-(trifluoromethyl)phenyl)urea hydrobromide of this invention.

The term “crystalline N-(4-(4-aminothieno[2,3-d]pyrimidin-5-yl)phenyl)-N′-(2-fluoro-5-(trifluoromethyl)phenyl)urea hydrobromide of this invention,” as used herein, means crystalline N-(4-(4-aminothieno[2,3-d]pyrimidin-5-yl)phenyl)-N′-(2-fluoro-5-(trifluoromethyl)phenyl)urea hydrobromide characterized, when measured at about 25° C. with radiation at 1.54178 Å, by a powder diffraction pattern with at least three peaks having respective 2θ values of about 6.1°, 8.4°, 10.4°, 12.5°, 13.3°, 14.3°, 15.9° 17.5°, 19.2° or 21.5°.

Unless stated otherwise, percentages herein are weight/weight (w/w) percentages.

The term “substantial crystalline purity,” as used herein, means at least about 95% crystalline purity, preferably about 97% crystalline purity, more preferably about 99% crystalline purity, and most preferably about 100% crystalline purity.

The term “crystalline purity,” as used herein, means percentage of a particular crystalline form of a compound in a sample which may contain amorphous form of the compound, one or more than one other crystalline forms of the compound other than the crystalline form of the compound of this invention, or a mixture thereof.

The term “substantial chemical purity,” as used herein, means about 95% chemical purity, preferably about 97% chemical purity, more preferably about 98% chemical purity, and most preferably about 100% chemical purity.

This invention is also meant to include mixtures comprising the crystalline N-(4-(4-aminothieno[2,3-d]pyrimidin-5-yl)phenyl)-N′-(2-fluoro-5-(trifluoromethyl)phenyl)urea hydrobromide of this invention in combination with an amorphous form of N-(4-(4-aminothieno[2,3-d]pyrimidin-5-yl)phenyl)-N′-(2-fluoro-5-(trifluoromethyl)phenyl)urea hydrobromide, one or more than one crystalline forms of N-(4-(4-aminothieno[2,3-d]pyrimidin-5-yl)phenyl)-N′-(2-fluoro-5-(trifluoromethyl)phenyl)urea hydrobromide other than the crystalline N-(4-(4-aminothieno[2,3-d]pyrimidin-5-yl)phenyl)-N′-(2-fluoro-5-(trifluoromethyl)phenyl)urea hydrobromide of this invention or mixtures thereof.

It is meant to be understood that each component of mixtures consisting essentially of two or more forms of N-(4-(4-aminothieno[2,3-d]pyrimidin-5-yl)phenyl)-N′-(2-fluoro-5-(trifluoromethyl)phenyl)urea hydrobromide may have varying degrees of chemical purity and that, in a preferred embodiment for the practice of this invention, in mixtures comprising different forms of N-(4-(4-aminothieno[2,3-d]pyrimidin-5-yl)phenyl)-N′-(2-fluoro-5-(trifluoromethyl)phenyl)urea hydrobromide, each component of the mixture is substantially chemically pure.

The term “solvent,” as used herein, means a liquid substance in which a compound is soluble or partially soluble enough at a given concentration to dissolve or partially dissolve the compound.

The term “anti-solvent,” as used herein, means a liquid in which a compound is insoluble enough at a given concentration to be effective for precipitating that compound.

Solvents and anti-solvents may be mixed with or without emulsification.

It is meant to be understood that, because many solvents and anti-solvents contain impurities, the level of impurities in solvents and anti-solvents for the practice of this invention, if present, are at a low enough concentration that they do not interfere with the intended use of the solvent in which they are present.

Causing a crystalline N-(4-(4-aminothieno[2,3-d]pyrimidin-5-yl)phenyl)-N′-(2-fluoro-5-(trifluoromethyl)phenyl)urea hydrobromide to exist in a mixture in which it has completely dissolved is known as nucleation.

For the practice of this invention, nucleation may be made to occur by means such as solvent removal, temperature change, solvent-miscible anti-solvent addition, solvent-immiscible anti-solvent addition, seed crystal addition of a crystalline N-(4-(4-aminothieno[2,3-d]pyrimidin-5-yl)phenyl)-N′-(2-fluoro-5-(trifluoromethyl)phenyl)urea hydrobromide, chafing or scratching the interior of the container, preferably a glass container, in which nucleation is meant to occur with an implement such as a glass rod or a glass bead or beads, or a combination of the foregoing.

For the practice of this invention, nucleation may be followed by crystal growth, accompanied by crystal growth, or followed and accompanied by crystal growth during which, and as a result of which, the percentage of N-(4-(4-aminothieno[2,3-d]pyrimidin-5-yl)phenyl)-N′-(2-fluoro-5-(trifluoromethyl)phenyl)urea hydrobromide increases.

It is meant to be understood that airborne seed crystals of a crystalline N-(4-(4-aminothieno[2,3-d]pyrimidin-5-yl)phenyl)-N′-(2-fluoro-5-(trifluoromethyl)phenyl)urea hydrobromide may also cause nucleation in a mixture comprising N-(4-(4-aminothieno[2,3-d]pyrimidin-5-yl)phenyl)-N′-(2-fluoro-5-(trifluoromethyl)phenyl)urea hydrobromide and solvent in which the N-(4-(4-aminothieno[2,3-d]pyrimidin-5-yl)phenyl)-N′-(2-fluoro-5-(trifluoromethyl)phenyl)urea hydrobromide has completely dissolved.

The term “seed crystal,” as used herein, means a particular crystalline form of a substance having mass. It is meant to be understood that such a crystal may be small enough to be airborne or invisible to the eye without means of detection.

The term “isolating” as used herein, means separating a crystalline N-(4-(4-aminothieno[2,3-d]pyrimidin-5-yl)phenyl)-N′-(2-fluoro-5-(trifluoromethyl)phenyl)urea hydrobromide and solvent, anti-solvent, or a mixture comprising solvent and anti-solvent. This is typically accomplished by means such as centrifugation, filtration with or without vacuum, filtration with positive pressure, distillation, evaporation or a combination thereof.

A therapeutically acceptable amount of a crystalline N-(4-(4-aminothieno[2,3-d]pyrimidin-5-yl)phenyl)-N′-(2-fluoro-5-(trifluoromethyl)phenyl)urea hydrobromide depends on recipient of treatment, disorder being treated and severity thereof, composition containing it, time of administration, route of administration, duration of treatment, its potency, its rate of clearance and whether or not another drug is co-administered. The amount of a crystalline N-(4-(4-aminothieno[2,3-d]pyrimidin-5-yl)phenyl)-N′-(2-fluoro-5-(trifluoromethyl)phenyl)urea hydrobromide used to make a composition to be administered daily to a patient in a single dose or in divided doses is from about 0.03 to about 200 mg/kg body weight. Single dose compositions contain these amounts or a combination of submultiples thereof.

A crystalline N-(4-(4-aminothieno[2,3-d]pyrimidin-5-yl)phenyl)-N′-(2-fluoro-5-(trifluoromethyl)phenyl)urea hydrobromide may be administered with or without an excipient. Excipients include but are not limited to, for example, encapsulating materials and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising and made with a crystalline N-(4-(4-aminothieno[2,3-d]pyrimidin-5-yl)phenyl)-N′-(2-fluoro-5-(trifluoromethyl)phenyl)urea hydrobromide to be administered orally in solid dosage form include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, mixtures thereof and the like. Excipients for preparation of compositions comprising and made with a crystalline N-(4-(4-aminothieno[2,3-d]pyrimidin-5-yl)phenyl)-N′-(2-fluoro-5-(trifluoromethyl)phenyl)urea hydrobromide to be administered ophthalmically or orally in liquid dosage forms include, for example, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water, mixtures thereof and the like. Excipients for preparation of compositions comprising and made with a crystalline N-(4-(4-aminothieno[2,3-d]pyrimidin-5-yl)phenyl)-N′-(2-fluoro-5-(trifluoromethyl)phenyl)urea hydrobromide to be administered osmotically include, for example, chlorofluorohydrocarbons, ethanol, water, mixtures thereof and the like. Excipients for preparation of compositions comprising and made with a crystalline N-(4-(4-aminothieno[2,3-d]pyrimidin-5-yl)phenyl)-N′-(2-fluoro-5-(trifluoromethyl)phenyl)urea hydrobromide to be administered parenterally include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water, mixtures thereof and the like. Excipients for preparation of compositions comprising and made with a crystalline N-(4-(4-aminothieno[2,3-d]pyrimidin-5-yl)phenyl)-N′-(2-fluoro-5-(trifluoromethyl)phenyl)urea hydrobromide to be administered rectally or vaginally include, but are not limited to, cocoa butter, polyethylene glycol, wax, mixtures thereof and the like.

The following examples are presented to provide what is believed to be the most useful and readily understood description of procedures and conceptual aspects of this invention.

### EXAMPLE 1

A mixture of 1-(4-nitrophenyl)ethanone (15 g), malononitrile (6 g), ammonium acetate (7 g) and acetic acid (10 mL) in benzene (200 mL) at reflux was stirred for 18 hours with azeotropic removal of water, cooled, poured into water, and extracted with ethyl acetate. The combined extracts were washed with water and brine and dried (MgSO₄), filtered and concentrated. The concentrate was flash chromatographed on silica gel with 25% ethyl acetate/hexanes.

### EXAMPLE 2

EXAMPLE 58A (4.14 g) in ethanol (200 mL) and THF (80 mL) at 25° C. was treated sequentially with sulfur (621 mg) and triethylamine (1.82 mg), stirred for 18 hours and filtered. The filtrant was absorbed onto silica and flash column chromatographed with 3:2 hexanes/ethyl acetate.

### EXAMPLE 3

EXAMPLE 2 (1.23 g) in formamide (20 mL) between 150° C. and 160° C. was stirred for 19 hours, cooled, and filtered.

### EXAMPLE 4

EXAMPLE 3 (500 mg) in THF (30 mL), water (15 mL), and ethanol (40 mL) at 50° C. was treated with iron powder (0.616 g), heated between 70° C. and 80° C. for two hours and filtered through diatomaceous earth (Celite®) while hot. The filtrant was washed with THF (10 mL) and ethanol and the combined filtrates were concentrated. The concentrate was partitioned between water and ethyl acetate and the aqueous phase was extracted three times with ethyl acetate. The combined extracts were washed with brine and dried (MgSO₄), filtered and concentrated to provide 432 mg of the desired product.

### EXAMPLE 5

EXAMPLE 4 (40 mg) in dichloromethane (3 mL) at 0° C. was treated with 1-fluoro-2-isocyanato-4-(trifluoromethyl)benzene (24 μL), stirred for 18 hours while gradually warming to 25° C. and filtered. The filtrant was dried under vacuum. ¹H NMR (300 MHz, DMSO-d₆) δ 9.40 (s, 1H); 8.98 (d, 1H); 8.63 (dd, 2.1 Hz, 1H); 8.35 (s, 1H); 7.63 (d, 2H); 7.55-7.39 (m, 5H).

### EXAMPLE 6

EXAMPLE 5 (3.0 g) in N,N-dimethylacetamide (9 mL) was treated with water (180 mL), stirred for 30 minutes and filtered. The filtrant was washed with water (50 mL total), stirred at reflux with absolute ethanol (30 mL) for one hour, cooled, treated with THF (10 mL), stirred again at reflux for 30 minutes, treated with 48% HBr in acetic acid (2.66 mL), stirred for another 15 minutes, treated with acetonitrile (40 mL), cooled down to 25° C., stirred for one hour, treated with acetonitrile (50 mL) to provide a solution from which solid began to slowly form, stirred at 25° C. for 16 hours and filtered. The filtrant was washed with acetonitrile (30 mL) and absolute ethanol (10 mL) and dried under vacuum at 60° C. for 25 hours.

A sample of crystalline N-(4-(4-aminothieno[2,3-d]pyrimidin-5-yl)phenyl)-N′-(2-fluoro-5-(trifluoromethyl)phenyl)urea hydrobromide of this invention for powder diffraction analysis was applied as a thin layer, with no prior grinding, to the analysis well of a Scintag×2 Diffraction Pattern System having the following parameters: x-ray source: Cu—Kα; range: 2.0° to 40.0° 2θ; scan rate: 1.2 degree per minute; step size: 0.02°; temperature: 25° C.; wavelength: 1.54178 Å (Cu—Kα).

The term “about” preceding a series of peak positions is meant to include all of the peak positions of the group which it precedes.

It is meant to be understood that relative intensities of peak heights in a PXRD pattern may vary and will be dependent on variables such as the temperature, size of crystal size or morphology, sample preparation, or sample height in the analysis well of the X-ray diffractometer.

It is also meant to be understood that peak positions may vary when measured with different radiation sources. For example, Cu—Kα₁, Mo—Kα, Co—Kα and Fe—Kα radiation, having wavelengths of 1.54060 Å, 0.7107 Å, 1.7902 Å and 1.9373 Å, respectively, may provide peak positions which differ from those measured with Cu—Kαradiation, which has a wavelength of 1.5478 Å.

The term “about” preceding a series of peak positions means that all of the peaks of the group which it precedes are reported in terms of angular positions (2θ) with an allowable variability of ±0.1° as specified by the U.S. Pharmacopeia, pages 1843-1884 (1995). The variability of ±0.1° is intended to be used when comparing two powder X-ray diffraction patterns. In practice, if a diffraction pattern peak from one pattern is assigned a range of angular positions (2θ) which is the measured peak position ±0.1° and if those ranges of peak positions overlap, then the two peaks are considered to have the same angular position. For example, if a peak from one pattern is determined to have a position of 5.2°, for comparison purposes the allowable variability allows the peak to be assigned a position in the range of 5.1°-5.3°. If a peak from another diffraction pattern has a peak position of 5.3°, for comparison purposes, the allowable variability allows the peak to be assigned a position in the range of 5.2°-5.4°. Because there is overlap between the two ranges of peak positions (i.e., 5.1°-5.3° and 5.2°-5.4°) the two peaks being compared are considered to have the same angular position.

The foregoing is meant to be illustrative of the invention and not intended to limit it to the disclosed embodiments. Variations and changes obvious to one skilled in the art are intended to be within the scope and nature of the invention as defined in the claims.

We claim:

1. Crystalline N-(4-(4-aminothieno[2,3-d]pyrimidin-5-yl)phenyl)-N′-(2-fluoro-5-(trifluoromethyl)phenyl)urea hydrobromide characterized, when measured at about 25° C. with radiation at 1.54178 Å, by a powder diffraction pattern with at least three peaks having respective 2θ values of about 6.1°, 8.4°, 10.4°, 12.5°, 13.3°, 14.3°, 15.9°, 17.5°, 19.2° or 21.5°.

2. Crystalline N-(4-(4-aminothieno[2,3-d]pyrimidin-5-yl)phenyl)-N′-(2-fluoro-5-(trifluoromethyl)phenyl)urea hydrobromide having substantial crystalline purity and characterized, when measured at about 25° C. with radiation at 1.54178 Å, by a powder diffraction pattern with at least three peaks having respective 2θ values of about 6.1°, 8.4°, 10.4°, 12.5°, 13.3°, 14.3°, 15.9°, 17.5°, 19.2° or 21.5°.

3. A composition comprising an excipient and crystalline N-(4-(4-aminothieno[2,3-d]pyrimidin-5-yl)phenyl)-N′-(2-fluoro-5-(trifluoromethyl)phenyl)urea hydrobromide characterized, when measured at about 25° C. with radiation at 1.54178 Å, by a powder diffraction pattern with at least three peaks having respective 2θ values of about 6.1°, 8.4°, 10.4°, 12.5°, 13.3°, 14.3°, 15.9°, 17.5°, 19.2° or 21.5°.

4. A method or treating a patient having a disease caused or exascerbated by upregulation or overexpression of protein tyrosine kinases comprising administering thereto a therapeutically effective amount of crystalline N-(4-(4-aminothieno[2,3-d]pyrimidin-5-yl)phenyl)-N′-(2-fluoro-5-(trifluoromethyl)phenyl)urea hydrobromide characterized, when measured at about 25° C. with radiation at 1.54178 Å, by a powder diffraction pattern with at least three peaks having respective 2θ values of about 6.1°, 8.4°, 10.4°, 12.5°, 13.3°, 14.3°, 15.9°, 17.5°, 19.2° or 21.5°.

5. A process for making a crystalline N-(4-(4-aminothieno[2,3-d]pyrimidin-5-yl)phenyl)-N′-(2-fluoro-5-(trifluoromethyl)phenyl)urea hydrobromide, said process comprising:
providing a mixture comprising N-(4-(4-aminothieno[2,3-d]pyrimidin-5-yl)phenyl)-N′-(2-fluoro-5-(trifluoromethyl)phenyl)urea, benzenesulfonic acid and solvent wherein said N-(4-(4-aminothieno[2,3-d]pyrimidin-5-yl)phenyl)-N′-(2-fluoro-5-(trifluoromethyl)phenyl)urea is completely dissolved in said solvent; and
causing crystalline aminothieno[2,3-d]pyrimidin-5-yl)phenyl)-N′-(2-fluoro-5-(trifluoromethyl)phenyl)urea hydrobromide to exist in said mixture, said N-(4-(4-aminothieno[2,3-d]pyrimidin-5-yl)phenyl)-N′-(2-fluoro-5-(trifluoromethyl)phenyl)urea hydrobromide, when isolated, characterized, when measured at about 25° C. with radiation at 1.54178 Å, by a powder diffraction pattern with at least three peaks having respective 2θ values of about 6.1°, 8.4°, 10.4°, 12.5°, 13.3°, 14.3°, 15.9°, 17.5°, 19.2° or 21.5°.

6. The process of claim 5 further comprising isolating said N-(4-(4-aminothieno[2,3-d]pyrimidin-5-yl)phenyl)-N′-(2-fluoro-5-(trifluoromethyl)phenyl)urea hydrobromide.

7. Crystalline N-(4-(4-aminothieno[2,3-d]pyrimidin-5-yl)phenyl)-N′-(2-fluoro-5-(trifluoromethyl)phenyl)urea hydrobromide prepared by the processes of claim 6.
